(19) United States
(12) Patent Application Publication
(10) Pub. No.: US 2016/0213754 A1
(43) Pub. Date: Jul. 28, 2016

(54) TRANSGLUTAMINASE 4 AS A PROSTATE SPECIFIC AUTOANTIGEN AND THE METHODS OF USE THEREOF

(71) Applicants: Nils LANDEGREN, Uppsala (SE); Olof KAMPE, Uppsala (SE); Michael SNYDER, Stanford, CA (US); Donald SHARON, Stanford, CA (US)

(72) Inventors: Nils LANDEGREN, Uppsala (SE); Olof KAMPE, Uppsala (SE); Michael SNYDER, Stanford, CA (US); Donald SHARON, Stanford, CA (US)

(21) Appl. No.: 15/023,434

(22) PCT Filed: Sep. 18, 2014
(86) PCT No.: PCT/US14/56239

§ 371 Date: Mar. 21, 2016

Related U.S. Application Data
(60) Provisional application No. 61/880,590, filed on Sep. 20, 2013.

(51) Int. Cl. A61K 38/45; C07K 16/40

(52) U.S. Cl. CPC A61K 38/45; C07K 16/40

(57) ABSTRACT

The disclosure relates to transglutaminase 4 as a prostate specific autoantigen, and methods for diagnosis of autoimmune polyendocrune syndrome type 1, autoimmune prostatitis in autoimmune polyendocrine syndrome type 1, and isolated autoimmune prostatitis. The disclosure further relates to use of transglutaminase 4 and transglutaminase 4 antibodies and antibody conjugates for the treatment of prostate cancers.

This application claims benefit of priority to U.S. Provisional Application Ser. No.61/880,590, filed Sep. 20, 2013, the entire contents of which are hereby incorporated by reference.

### BACKGROUND OF THE INVENTION

I. Field of the Invention

The present invention relates to the fields of medicine, diagnostics and oncology. More particular, transglutaminase 4 has been identified as a new prostate specific autoantigen, and methods for diagnosis of autoimmune polyendocrine syndrome type 1, autoimmune prostatitis in autoimmune polyendocrine syndrome type 1, and isolated autoimmune prostatitis, as well as the treatment prostate cancers, are described.

II. Related Art

Autoimmune polyendocrine syndrome type 1 (APS-1) (Online Mendelian Inharitance in Man, number 240300) is a rare potentially fatal syndrome with both endocrine and non-endocrine components. The syndrome is due to mutations in the Aire (autoimmune regulator) gene on chromosome 21 and displays recessive inheritance. This rare disease has been instrumental in unravelling the molecular mechanisms of central tolerance and negative selection APS-1 is the most studied human model for tissue-specific autoimmune disease and a number of novel autoantigens have been identified using sera from patients with APS-1, e.g., side-chain cleavage enzyme in isolated autoimmune gonadal failure.

### SUMMARY OF THE INVENTION

The present inventors have identified transglutaminase 4 (TGM4) as a novel major autoantigen of the prostate gland and demonstrate that the expression of TGM4 in the prostate gland at puberty in males with APS-1 is an absolute prerequisite for autoantibody formation representing the first demonstration of prostate autoimmunity in APS-1. TGM4 autoantibodies were specifically detected in the male and only after the age of pubertal debut.

A first aspect of the present invention provides methods for the diagnosis of APS-1, of autoimmune prostatitis in APS-1, and of isolated autoimmune prostatitis. The methods comprise detection of autoantibodies specific for TGM4 in a sample obtained from a subject. The subject can be a human. The sample can be a blood sample, such as serum or plasma, a urine sample, a semen sample, a prostate biopsy or prostatic fluid.

Antibodies specific or selective for TGM4 can be detected using an immunoassay, such as ELISA, RIA, or radioimmunoprecipitaton assays, by surface plasmon resonance, or by electrochemiluminescence.

A second aspect of the present invention provides use of TGM4 for the treatment of prostate cancer.

In one embodiment the method comprises administering a pharmaceutical composition comprising a therapeutically active amount of TGM4 or a fragment thereof to a patient in need of such treatment. The fragment may be 10, 15, 20, 25, 30, 35, 40, 45, 50, 60, 75, 100, 150, 200, 250, 300, 350 or 400 consecutive residues of TGM4.

Accordingly, the second aspect of the present invention provides a pharmaceutical composition comprising therapeutically effective amount of TGM4 or a fragment thereof for use in the treatment of prostate cancer.

Put another way, the second aspect of the present invention provides for use of TGM4 or a fragment thereof for use in the manufacture of a pharmaceutical composition for treatment of prostate cancer.

A third aspect of the present invention provides use of antibodies specifically or selectively binding to TGM4 for the treatment of prostate cancer.

In one embodiment the method comprises administering a pharmaceutical composition comprising therapeutically effective amount of an antibody, an antibody fragment, a bispecific antibody, or an antibody conjugate specifically or selectively binding to TGM4 to a patient in need of such treatment.

Accordingly, the third aspect of the present invention provides a pharmaceutical composition comprising therapeutically effective amount of an antibody, an antibody fragment, a bispecific antibody, or an antibody conjugate specifically or selectively binding to TGM4 for use in the treatment of prostate cancer.

Put another way, the third aspect of the present invention provides for use of an antibody, an antibody fragment, a bispecific antibody, or an antibody conjugate specifically or selectively binding to TGM4 for use in the manufacture of a pharmaceutical composition for treatment of prostate cancer.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed.

FIG. 1. Protein array screening identifies TGM4 as an autoantigen. Human protein arrays were screened with serum from 51 APS-1 patients and 21 healthy control subjects. Autoantibodies to TGM4 were detected in 16 APS-1 patients and in one healthy subject (cutoff =average of the healthy +3SD).

FIG. 2. TGM4 autoantibodies are APS-1-specific. TGM4 autoantibodies were measured in 93 APS-1 patients, 130 healthy control subjects and 80 autoimmune disease control subjects (20 patients in each group). 27 APS-1 patients (29%) demonstrated TGM4-specific antibodies, while autoantibodies were absent in all of the healthy and disease control subjects. TGM4 autoantibodies were measured in serum by immunoprecipitation and the upper limit of the normal range was defined as an index-value of 10.

FIGS. 3A-C. Transglutaminase-related immune-disorders and molecular specificity of TGM4 autoantibodies. (FIG. 3A) Protein sequence similarity between members of the human transglutaminase family is represented by a phylogram (created with

ClustalW). The dominating tissue distribution of each transglutaminase (as investigated and reviewed elsewhere^{4,16,24-25}) and autoimmune diseases characterized by autoantibodies to respective transglutaminase are also displayed³⁻⁶. To assess the specificity of translutaminase autoantibodies in APS-1 and celiac disease, the inventors precipitated radio-labeled TGM4 (FIG. 3B) and TGM2 (FIG. 3C) with sera from 93 APS-1 patients, 50 celiac disease patients and 130 healthy control subjects. APS-1 patient antibodies only reacted with TGM4 and celiac disease patients only reacted with TGM2.

FIGS. 4A-C. TGM4 autoantibodies are male-specific and appear after the age of pubertal debut. TGM4 autoantibodies were measured among 47 male and 46 female APS-1 patients. TGM4-specific antibodies were detected in 26 (56%) male patients and only in one female patient (FIG. 4A). All of the TGM4-reactive males were older than 12 years (FIG. 4B), and none of the established APS-1 autoantigens demonstrated similar age dependent distribution (Table 1). TGM4 autoantibodies were measured in consecutive samples from six TGM4-reactive male patients, and were first detected by the age of 12, 13, 14, 15, 16 and 16 years respectively (FIG. 4C). Each blood sample is represented by a dot along the timeline, and TGM4 autoantibody-positivity is marked in black, negative in grey. TGM4 autoantibodies were measured in serum by immunoprecipitation and the upper limit of the normal range was defined as an index-value of 10.

FIG. 5. Male Aire-/-mice develop lymphocytic infiltration and destruction of the prostate in association with autoantibodies to murine TGM4.

FIG. 6. TGM4 autantibodies detected on the human protein array. Human protein arrays were screened with sera from 51 APS-1 patients and 21 healthy control subjects. Autoantibodies to TGM4 were detected in 16 APS-1 patients and in one healthy subject. The dotted line represents the upper limit of the normal range, defined as average of the healthy +3SD (corresponding to a fluorescence signal of 466). The TGM4-antibody signal in the positive female APS-1 patient and positive healthy subject sera were however low compared with the average of the TGM4-reactive male group (5.8% and 3.7% respectively).

FIG. 7. Validation of TGM4 autoantibodies by immunoprecipitation in the APS-1 discovery cohort (n=51). All patients demonstrating TGM4-specific autoantibodies in the protein array screen were confirmed positive (n=16). Among the patients classified as TGM4-negative in the protein array screen (n=35), one patient was found reactive and the remaining patients were confirmed negative.

FIG. 8. TGM4 autoantibodies in the APS-1 discovery and replication cohorts. TGM4 autoantibodies were detected in 17 out of 51 APS-1 patients in the discovery cohort. A replication cohort of 42 APS-1 patients was investigated, where another 10 TGM4-reactive patients were identified. TGM4 autoantibodies were measured in serum by immune-precipitation and the upper limit of the normal range was defined as an index-value of 10.

FIG. 9. TGM4 autoantibodies appear after the age of pubertal debut. TGM4 autoantibodies were measured in consecutive samples from six TGM4-reactive male patients, and were first detected by the age of 12, 13, 14, 15, 16 and 16 years respectively. TGM4 autoantibodies were measured in serum by immunoprecipitation and the upper limit of the normal range was defined as an index-value of 10.

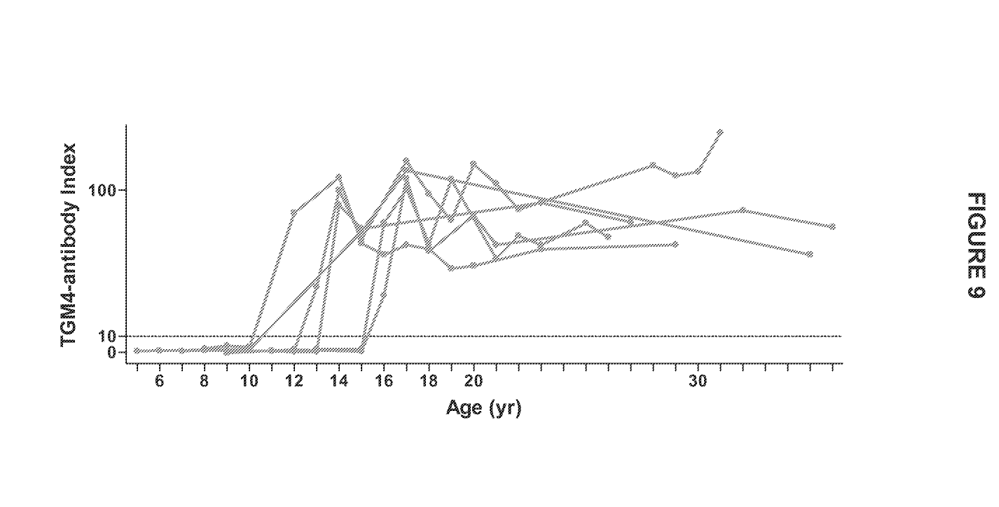

### DETAILED DESCRIPTION OF THE INVENTION

In one aspect, the present invention provides methods for the diagnosis of APS-1 and autoimmune prostatitis. The methods comprise detection of autoantibodies specifically or selectively binding to TGM4 in a sample obtained from a subject. The subject can be a human. The sample can be a blood sample, such as serum or plasma, a urine sample, a semen sample, a prostate biopsy or prostatic fluid.

Antibodies specifically or selectively binding to TGM4 can be detected using an immunoassay, such as ELISA, or RIA, by surface plasmon resonance, or electrochemiluminescence. The “immunoassay” used to detect autoantibodies specifically or selectively binding to TGM4 according to the invention may be based on standard techniques known in the art. In a particular embodiment the immunoassay may be an ELISA.

ELISAs are generally well known in the art. In a typical ELISA the TGM4 antigen is immobilised on a solid surface (e.g., the wells of a standard microtiter assay plate, or the surface of a microbead or a microarray) and a sample comprising the sample to be tested for the presence of autoantibodies specifically binding to TGM4 is brought into contact with the immobilised antigen. Any autoantibodies of the desired specificity present in the sample will bind to the immobilised antigen. The bound antibody/antigen complexes may then be detected using any suitable method. In one embodiment, a labelled secondary anti-human immunoglobulin antibody, which specifically recognizes an epitope common to one or more classes of human immunoglobulins, is used to detect the antibody/antigen complexes. Typically the secondary antibody will be anti-IgG or anti-IgM. The secondary antibody is usually labelled with a detectable marker, typically an enzyme marker such as, for example, peroxidase or alkaline phosphatase, allowing quantitative detection by the addition of a substrate for the enzyme which generates a detectable product, for example a coloured, chemiluminescent or fluorescent product. Other types of detectable labels known in the art may be used.

In a radioimmunoprecipitation assay the antigen is typically produced in vitro by in vitro transcription of a specific plasmid containing the cDNA sequence of TGM4 or a fragment thereof with suitable promotors, e.g., T7, T3 och SP-6, followed by in vitro translation in the precens of a components from a reticulocyte lysate and a radioactive amino acid, e.g., ³⁵S-methionine. The resulting radioactive proteins are then incubated with patient sera, the antibodies collected by Protein A or G bound to a matrix, antibodies against IgG and or IgM and or IgA, collected, washed and the bound radioactivity analysed.

Full-length TGM4 or one or more peptides derived from the amino acid sequence of TGM4 can be used as antigen in the detection of autoantibodies specifically binding to TGM4. The amino acid sequence of TGM4 can be found in GenBank Accession No. NP _003232 or SwissProt/UniProt Accesion No. P49221 (TGM4_HUMAN).

A second aspect of the present invention provides use of TGM4 for the treatment of prostate cancer. In one embodiment the method comprises administering a pharmaceutical composition comprising a therapeutically active amount of TGM4 or a fragment thereof to a patient in need of such treatment. Accordingly, the second aspect of the present invention provides a pharmaceutical composition comprising therapeutically effective amount of TGM4 or a fragment thereof for use in the treatment of prostate cancer with or without and adjuvant or other immunostimulatory molecules or cells. Put another way, the second aspect of the present invention provides for use of TGM4 or a fragment thereof for use in the manufacture of a pharmaceutical composition for treatment of prostate cancer. In one embodiment, use of TGM4 or a fragment thereof for the treatment of prostate cancer according to the second aspect of the invention, is intended to evoke an immune response to TGM4 on prostate cancer cells in the patient in need of such treatment. The immune response can be a B-cell response and/or a T-cell response. The fragment of TGM4 may be an immunological active fragment, an epitope.

The pharmaceutical composition comprising a therapeutically active amount of TGM4 or a fragment thereof may also comprise an adjuvant. Adjuvants include any compound or compounds that act to increase an immune response to the TGM4 antigen, thereby reducing the quantity of antigen necessary in the vaccine, and/or the frequency of administration necessary to generate a protective immune response. Adjuvants can include for example, emulsifiers, muramyl dipeptides, pyridine, aqueous adjuvants such as aluminum hydroxide, chitosan-based adjuvants, and any of the various saponins, oils, and other substances known in the art, such as Amphigen, LPS, bacterial cell wall extracts, bacterial DNA, CpG sequences, synthetic oligonucleotides, natural or synthetic lipids binding to the CD1d molecule expressed on NKT cells, and combinations thereof.

A third aspect of the present invention provides use of antibodies directed to TGM4 for the treatment of prostate cancer. In one embodiment the method comprises administering a pharmaceutical composition comprising therapeutically effective amount of an antibody, an antibody fragment, a bispecific antibody, or an antibody conjugate specifically or selectively binding to TGM4 to a patient in need of such treatment. Accordingly, the third aspect of the present invention provides a pharmaceutical composition comprising therapeutically effective amount of an antibody, an antibody fragment, a bispecific antibody, or an antibody conjugate specifically or selectively binding to TGM4 for use in the treatment of prostate cancer. Put another way, the third aspect of the present invention provides for use of an antibody, an antibody fragment, a bispecific antibody, or an antibody conjugate specifically or selectively binding to TGM4 for use in the manufacture of a pharmaceutical composition for treatment of prostate cancer.

### I. ANTIBODIES

The term “antibody or antibody fragment” as referred to herein include whole antibodies and any antigen binding fragment referred to as “antigen-binding portion” or single chains thereof.

An “antibody” refers to a glycoprotein comprising at least two heavy (H) chains and two light (L) chains inter-connected by disulfide bonds, or an antigen binding portion thereof or a deglycosylated variant of this. Each heavy chain is comprised of a heavy chain variable region (abbreviated herein as V_{H}) and a heavy chain constant region. The heavy chain constant region is comprised of three domains, C_{H}1, C_{H}2 and C_{H}3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion”, as used herein, refers to one or more fragments of an antibody that retain the ability to specifically bind to an antigen (e.g. TGM4). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H}1 domains; (ii) a F(ab′)2 fragment, a bivalent fragment comprising two Fab fragments linked by a disulfide bridge at the hinge region; (iii) a Fab′ fragment, which is essentially an Fab with part of the hinge region; (iv) a Fd fragment consisting of the V_{H} and C_{H}1 domains; (v) a Fv fragment consisting of the V_{L} and V_{H} domains of a single arm of an antibody, (vi) a dAb fragment which consists of a V_{H} domain; (vii) an isolated complementarity determining region (CDR); and (viii) a nanobody, a heavy chain variable region containing a single variable domain and two constant domains. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody,” as used herein, is intended to refer to an antibody that is substantially free of other antibodies having different antigenic specificities (e.g., an isolated antibody that specifically binds TGM4 is substantially free of antibodies that specifically bind antigens other than TGM4). An isolated antibody that specifically binds TGM4 may, however, have cross-reactivity to other antigens, such as TGM4 molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared there from (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The term “bispecific antibody” is intended to refer to antibodies which have been engineered with dual specificity, consisting of two single-chain variable fragments (scFvs) of different antibodies. Bispecific antibodies include the Bi-specific T-cell engagers (BiTEs) wherein one of the scFvs binds to T cells (e.g. via the CD3 receptor), and the other to a tumor cell via a tumor specific antigen (e.g. TGM4 on a prostate cell).

The term “antibody conjugate” is intended to refer to antibodies or antibody fragments linked, via a stable, chemical, linker with labile bonds, to a biological active cytotoxic (anticancer) payload or drug.

### II. PHARMACEUTICAL COMPOSITIONS AND ROUTES OF ADMINISTRATION

Pharmaceutical compositions of the present invention comprise an effective amount of one or more candidate substance or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one candidate substance or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The candidate substance may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, subcutaneously, subconjunctival, intravesicularlly, mucosally, intrapericardially, intraumbilically, intraocularally, orally, locally, via inhalation (e.g., aerosol inhalation), via injection, via infusion, via continuous infusion, via localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The candidate substance may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines

In certain embodiments the candidate substance is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain particular embodiments, an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### III. EXAMPLES

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example 1

### Material & Methods

APS-1 patient samples and clinical characterization. APS-1 serum samples were collected from 93 Finnish, Norwegian and Swedish patients. All individuals met the clinical diagnostic criteria for APS-1 requiring two of the hallmark components; chronic mucocutaneous candidiasis, hypopararthyroidism and adrenal failure, or at least one of the hallmark components in siblings or children of APS-1 patients. Most of the patients also demonstrated typical Aire gene mutations (Table 1). Chronic mucocutaneous candidiasis was defined as candida infection of the oral mucosa, skin or nails for a period of more than three months. Hypoparathyroidism was defined as plasma calcium concentration below 2.15 and elevated plasma phosphate concentration in combination with normal or low parathormone (PTH) concentration and normal renal function. Adrenal failure was defined as sub-normal serum cortisol in combination with elevated plasma adrenocorticotropic hormone (ACTH) concentration or deficient response to synthetic ACTH stimulation test (failure to reach 550 nmol/L in 30 or 60 min) The patients had also been diagnosed with additional manifestations including alopecia, hypogonadism, vitiligo, insulin dependent diabetes mellitus, malabsorption and pernicious anemia. All patients had given their informed consent for participation.

Healthy and autoimmune disease control subjects. Serum samples were collected from patients with Addison's disease, primary Sjögren's syndrome, type 1 diabetes mellitus, autoimmune thyroiditis and tissue transglutaminase autoantibody positive patients under investigation for celiac disease. Blood donors were used as healthy control subjects. All control subjects were treated anonymously.

TABLE 1
Characterization of TGM4 positive and TGM4 negative APS-1 patients

| TGM4 | | | | | |
|---|---|---|---|---|---|
| autoantibody | | | Age | | |
| IP | Array | Sex | (yr) | APS-1 components | AIRE gene mutations |
| Pos | Pos | M | 25 | C, HP, AF, AI, V | R257X/R257X |
| Pos | Pos | M | 17 | C, HP, AF, M | R257X/R257X |
| Pos | Pos | M | 50 | C, AF, IDDM, M, PA | R257X/R257X |
| Pos | Pos | M | 34 | C, HP, AF, HG | R257X/R257X |
| Pos | Pos | M | 44 | C, HP, AF, Al, M, AH | R257X/R257X |
| Pos | Pos | M | 32 | C, HP, AF, IDDM | R257X/R257X |
| Pos | Pos | M | 59 | C, HP, AF, PA | R257X 1086/1097del |
| Pos | Pos | M | 24 | C, AF | R257X/R257X |
| Pos | Pos | M | 24 | C, AF, Al | R257X/R257X |
| Pos | Pos | M | 25 | C, HP, AF, Al, V | — |
| Pos | Pos | M | 30 | C, HP, AF, HG, C, IDDM | 967_979del/R257X |
| Pos | Pos | M | 16 | C, HP, AF, PA | c967-979del13/c967-979del13 |
| Pos | Pos | M | 58 | HP, AF, Al, V | c769C > T/c769C > T |
| Pos | Pos | M | 26 | C, HP, AF | c967-979del13/c967-979del13 |
| Pos | Pos | M | >30 | C, HP, AF, Al | — |
| Pos | Pos | M | 16 | C, AF | R257X/R257X |
| Pos | Neg | M | 24 | C, AF, Al, AH | — |
| Pos | — | M | 43 | HP, AF | c.769C > T/c.1279-G > C |
| Pos | — | M | >30 | — | — |
| Pos | — | M | 61 | C, HP, AF, Al, V | c967-979del13/c967-979del13 |
| Pos | — | M | 22 | C, HP | c1163_1164insA/c.967- |
| | | | | | 979del13 |
| Pos | — | M | 33 | C, HP, AF, Al, IDDM | R257X/R257X |
| Pos | — | M | 20 | C, HP, AF, IDDM | R257X/R257X |
| Pos | — | M | 48 | C, AF, A | R257X/R257X |
| Pos | — | M | 48 | C, HP, HG, V | — |
| Pos | — | M | 31 | C, HP, A, V | R257X/R257X |
| Pos | Pos | F | 42 | C, HP, AF, HG, V, IDDM, M | R257X/R257X |
| Neg | Neg | M | 29 | C, HP | R257X/R257X |
| Neg | Neg | M | 50 | C, HP, AF, HG, PA | — |
| Neg | Neg | M | 25 | C, AF | R257X/R257X |
| Neg | Neg | M | 27 | C, HP, AF, HG, AP | R257X/R257X |
| Neg | Neg | M | 40 | C, HP, AF, HG | R257X/R257X |
| Neg | Neg | M | 27 | C, HP, AF, HG, Al | R257X/X546C |
| Neg | Neg | M | 16 | C, HP, AH | R257X x2 |
| Neg | Neg | M | 44 | C, HP, AF, HG | c967-979del13/c967-979del13 |
| Neg | Neg | M | 19 | C, HP, AF, M | c967-979del13/c967-979del13 |
| Neg | — | M | 25 | C, AF, IDDM, M, AH, PA | — |
| Neg | — | M | 15 | C, HP, AF, M | — |
| Neg | — | M | 19 | C, HP, HG, M | — |
| Neg | — | M | 55 | C, HP, AF, Al | c22C > T/c402delC |
| Neg | — | M | 47 | C, HP, V, IDDM | c879 + 1G > A/c879 + 1G > A |
| Neg | — | M | 25 | C, HP, AF, Al, IDDM | |
| Neg | — | M | 11 | C, HP, AF | R257X/R257X |
| Neg | — | M | 12 | C, HP, AF, IDDM | R257X/R257X |
| Neg | Neg | F | — | C, HP, AF, V | R257X? |
| Neg | Neg | F | — | C, HP, AF, M, AH | A21V? |
| Neg | Neg | F | — | C, HP, HG, Al, V | — |
| Neg | Neg | F | 20 | C, HP, AF, V, PA | R257X/R257X |
| Neg | Neg | F | — | C, HP, | R257X/R257X |
| Neg | Neg | F | 21 | C, HP, AF, HG, AH | R257X/R257X |
| Neg | Neg | F | 36 | C, HP, AF, Al, V, M | R257X/R257X |
| Neg | Neg | F | 14 | C, HP, AF, AH | R257X/R257X |
| Neg | Neg | F | 33 | C, HP, AF, V | R257X/R257X |
| Neg | Neg | F | 32 | C, HP, AF, HG, IDDM, M, PA | R257X/R257X |
| Neg | Neg | F | 37 | C, HP, AF, HG, IDDM, M | R257X/R257X |
| Neg | Neg | F | 30 | C, HP, AF, HG | R257X C311Y |
| Neg | Neg | F | 33 | C, AF, HG | R257X C311Y |
| Neg | Neg | F | 14 | C, HP, AF, HG, Al, M, AH | R257X/R257X |
| Neg | Neg | F | 12 | C, HP, AF, AH | R257X/R257X |
| Neg | Neg | F | 39 | C, HP, AF, HG, V | R257X/R257X |
| Neg | Neg | F | 25 | C, HP, AF, HG, Al | R257X/R257X |
| Neg | Neg | F | 17 | C, HP, AF, HG, M, AH | R257X/R257X |
| Neg | Neg | F | 49 | C, HP, AF, HG, M | c967-979del13/c967-979del13 |
| Neg | Neg | F | 48 | C, HP, AF, HG | c967-979del13/c967-979del13 |
| Neg | Neg | F | 30 | C, HP, V | c967-979del13/c967-979del13 |
| Neg | Neg | F | 54 | C, HP, AF, HG, Al | c.769C > T/c.1336T > G |
| Neg | — | F | — | C, AF, M, AH | R257X/R257X |
| Neg | — | F | — | C, HP, PA | — |
| Neg | — | F | — | C, HP, AF, M, AH | — |
| Neg | — | F | — | C, HP, M | — |
| Neg | — | F | — | C, HP, | — |
| Neg | — | F | — | C, HP, HG, V, AH | — |
| Neg | — | F | — | C, HP, AF, HG, M, AH, PA | — |
| Neg | — | F | 16 | C, HP, AF | c769C > T/c1242_1234insA |
| Neg | — | F | 20 | C, AF | c769C > T/c1242_1234insA |
| Neg | — | F | 25 | C, AF, V, PA | c769C > T/c1242_1234insA |
| Neg | — | F | 39 | C, HP, AF, HG, Al | c1244_1245insC/ |
| | | | | | c.1244_1245insC |
| Neg | — | F | 32 | C, AF | c879 + 1G > A/c879 + 1G > A |
| Neg | — | F | 46 | C, HP, HG, Al | c22C > T/c209T > C |
| Neg | — | F | 36 | C, HP, AF, HG | R257X/R257X |
| Neg | — | F | 9 | C, HP, | R257X/R257X |
| Neg | — | F | 37 | C, HP, | R257X/R257X |
| Neg | — | F | 27 | C, HP, AF, HG, Al, PA | R257X/R257X |
| Neg | — | F | 28 | C, HP, AF, HG, Al, M, PA | R257X/R257X |
| Neg | — | F | 40 | C, HP, AF | R257X/R257X |
| Neg | — | F | 41 | C, HP, AF, HG | R257X/R257X |
| Neg | — | F | 20 | C, HP, AF, HG, Al, V, IDDM, PA | R257X/X546C |
| Neg | — | F | 15 | C, HP, AH | 1163\hat{ }1164insA/R257X |
| Neg | — | F | 10 | C, HP, AF, Al | R257X/R257X |

TGM4 autoantibodies were detected by immunoprecipitation (IP) and by the protein array (array). The upper limit of the normal range was defined as an index-value of 10 for the immunoprecipitation assay (IP) and as three standard deviations above the average of the healthy controls for the protein array. Disease components include candidiasis (C), hypoparathyroidism (H), adrenal failure (AF), alopecia (Al), hypogonadism (HG) (Al), vitiligo (V), insulin dependent diabetes mellitus (IDDM), malabsorption (M), autoimmune hepatitis (AH) and pernicious anemia (PA). Missing data is represented by a line (—).

Ethical considerations. The project was granted by ethical boards in Helsinki, Bergen and Uppsala.

Protein array screening. Protein arrays were probed and scanned as described in Invitrogens protocol for “Immune Response BioMarker Profiling”, and is explained in brief. Arrays, affinity reagents and blocking buffer were purchased from Life Technology Inc.;

ProtoArray® Human Protein Microarray v5.0 (PAH0525020, Life Technology), Alexa Fluor® 647 Goat Anti-Human IgG (A21445, Invitrogen), 10× Synthetic Block (PA017, Invitrogen). APS-1 patient (n=51) and healthy blood donor (n=21) serum samples were diluted 1:2000 in washing buffer. Incubations and washing steps were performed in a 4-chamber tray on 50 rpm rotation, at 4° C. Arrays were incubated in blocking buffer for 1 hour, washed for 5 min, and then incubated for 90 min in 5 ml of diluted serum. After 5×5 minutes washing, the arrays were incubated for 90 min with Alexa Fluor® 647 goat anti-human IgG antibody 1 mg/ml. The arrays were then washed for 5×5 minutes, submerged in distilled water and dried in a centrifuge at 200 ×g for 1 min. Arrays were scanned in a fluorescent microarray scanner. The GenePix® Pro microarray data acquisition software was used for alignment. The ProtoArray® Prospector v5.2 software was used to identify protein array targets that differed in signal intensity between patients and controls.

TGM4 immunoprecipitation assay. Serum autoantibodies were detected by immunoprecipitation with radio-labeled as previously reoprted²³, and is described briefly. Human TGM4 cDNA (SC303287, Origene) was cloned into pTNT-vector (L5610, Promega) and translated in vitro in the presence of ³⁵S-Methionine (Promega TNT Systems). Immunoprecipitation was conducted in 96 well filtration plates (Millipore). A positive standard represented by an APS-1 patient serum with TGM4-specific autoantibodies and a negative standard (4% BSA) were included to each plate. All serum samples were analyzed in duplicate. 40.000 counts per minute (CPM) of radiolabeled TGM4 protein and 2.5 μl of serum sample were added to each well, and incubated over night. Serum antibodies were then immobilized to protein A Sepharose (GE Health Care) during 1.5 h incubation. The plates were washed multiple times, dried and lastly was scintillation solution added. The radioactivity was measured in a micro-beta counter (Wallac). Index values were calculated according to the following: (sample value/negative standard)/(positive standard value/negative standard value)×100. Autoantibodies to tissue transglutaminase (TGM2), 21-hydroxylase (21OH), GAD65, 17-hydroxylase (17OH), side chain cleavage enzyme (SCC), tyrosine hydroxylase (TH), tryptophan hydroxylase, aromatic L-amino acid decarboxylase (AADC) and cytochrome P450 1A2 (CYP1A2), were analyzed in the same manner.

### Example 2

### Results

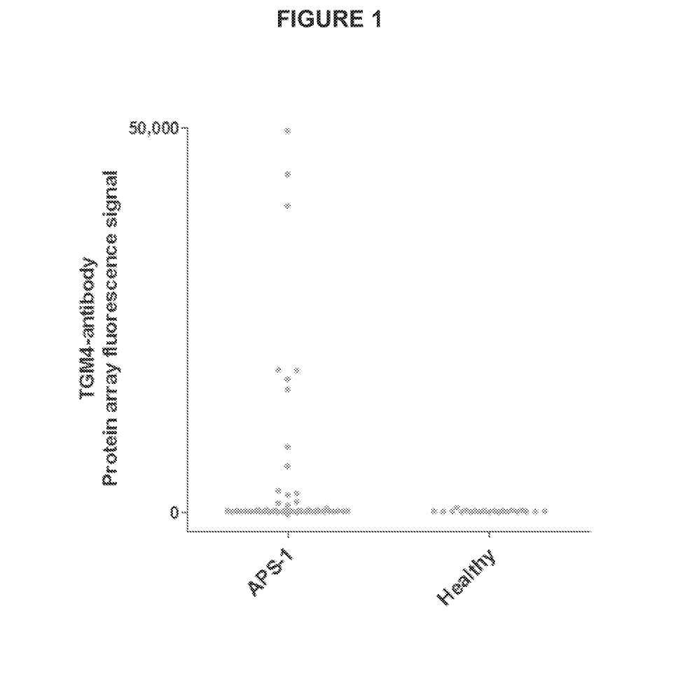

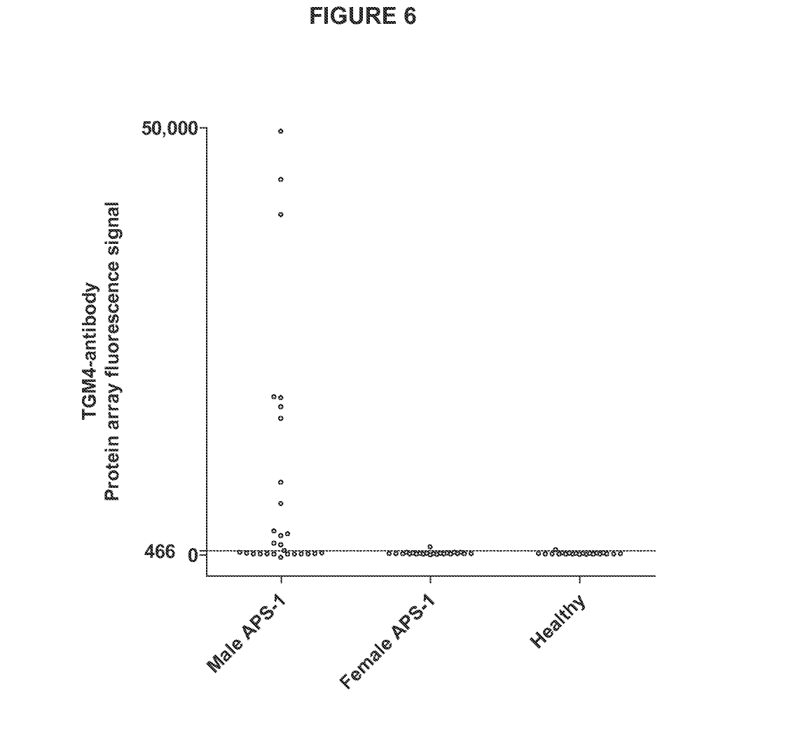

Protein array screening identifies tranglutaminase 4 (TGM4) as a novel autoantigen. Autoimmune manifestations in APS-1 are associated with circulating autoantibodies to proteins that are specifically expressed in the affected tissues¹⁻². A proteome-wide autoantibody screen in APS-1 were performed to identify novel autoantigens and unrecognized target organs. Human protein arrays containing over 9000 targets (Protoarray® Life Technologies) were probed with sera from 51 APS-1 patients and 21 healthy control subjects, and target-specific IgG autoantibodies were detected. More than 10 established APS-1 autoantigens were represented in the protein array panel, and all of these were confirmed as patient-specific targets. In addition, prostate-specific TGM4 was identified as a novel autoantigen. TGM4 autoantibodies were detected on the protein array in 16 out of 51 APS-1 patients, as compared with only one borderline positive out of 21 healthy subjects (cut off =average of the healthy+3 SD) (FIG. 1, FIG. 6).

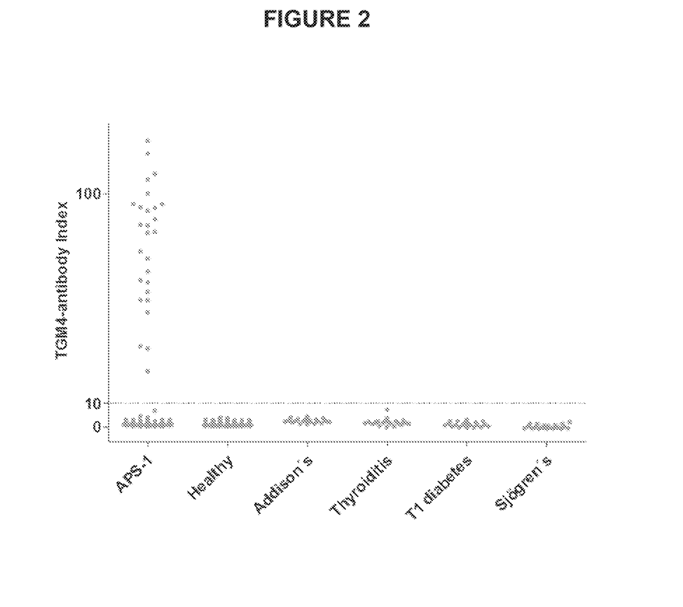

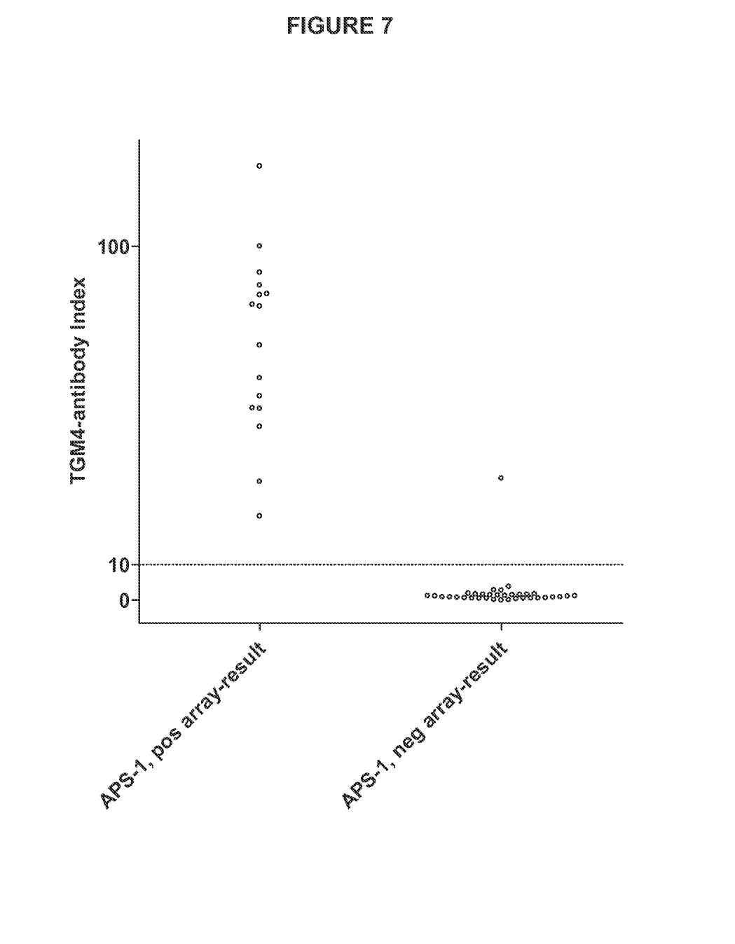

TGM4 autoantibodies are APS-1-specific. To confirm TGM4 as a valid autoantigen, an extended cohort of 93 APS-1 patients and over 250 healthy and autoimmune disease control subjects were screened for TGM4-specific antibodies. Radio-labeled TGM4 protein was produced in vitro using a modified pTNT-vector and precipitated with patient and control sera. Re-investigation of the discovery cohort of 51 APS-1 patients confirmed TGM4 autoantibodies in all 16 patients classified as reactive on the array. One additional TGM4-reactive patient was also identified, while the remaining 34 patients were confirmed negative (Table 1, FIG. 7). A replication cohort of 42 APS-1 patients was screened for TGM4 autoantibodies, where another 10 reactive patients were identified (Table 1, FIG. 7). Thereby 27 out of 93 APS-1 patients demonstrated TGM4-specific antibodies (FIG. 2).

To determine the clinical specificity of TGM4 autoantibodies, 135 healthy subjects and a selection of autoimmune diseases including Addison's disease, autoimmune thyroiditis, type 1 diabetes mellitus and Sjogren's syndrome (20 patients in each group) were screened. TGM4 autoantibodies were absent in all of the healthy and disease control subjects (FIG. 2). As a disease biomarker, TGM4 autoantibodies thereby demonstrated 29% sensitivity and 100% specificity for APS-1.

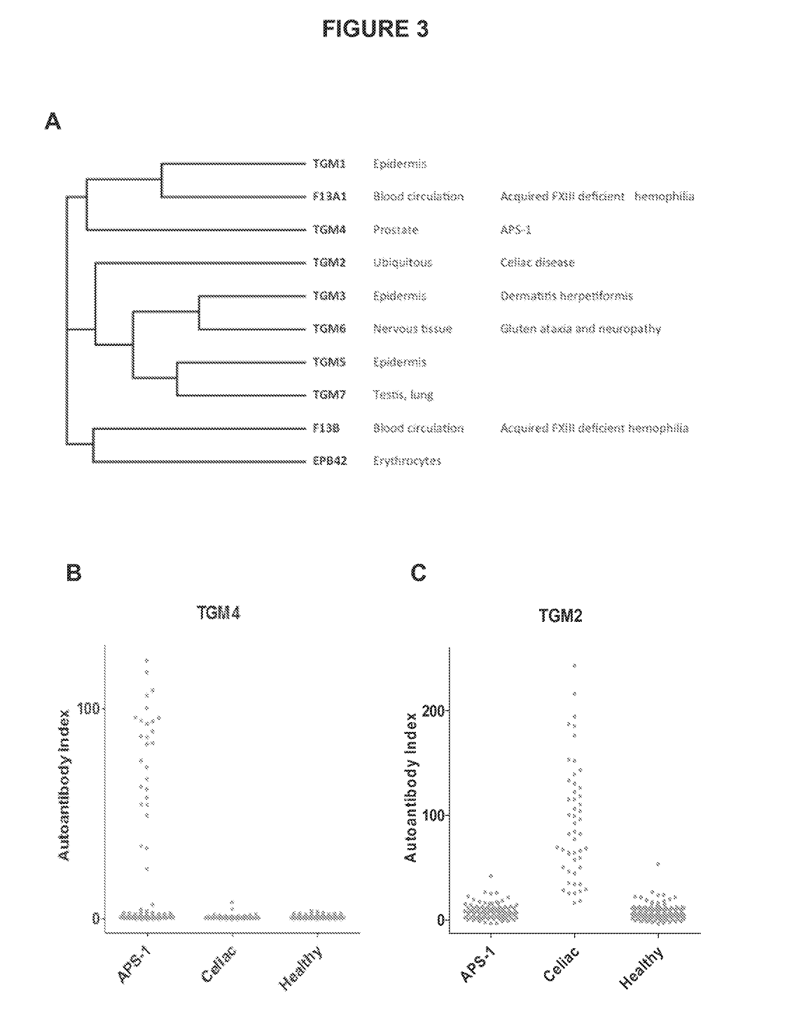

Members of the transglutaminase family have been recognized as autoantibody targets in a heterogeneous selection of autoimmune disorders³⁻⁶ (FIG. 3A). Tissue transglutaminase (TGM2) is the major autoantigen in celiac disease⁵ and display 33% protein sequence identity with TGM4 (protein sequences of human TGM4 and TGM2 were aligned using the Basic Local Alignment Search Tool). To assess the specificity of transglutaminase autoantibodies in APS-1 and celiac disease, radio-labeled TGM4 and TGM2 protein were immunoprecipitated with sera from the APS-1 patients and a cohort of 50 celiac disease patients. APS-1 patients exclusively reacted with TGM4 while celiac disease patients only recognized TGM2 (FIG. 3B-C).

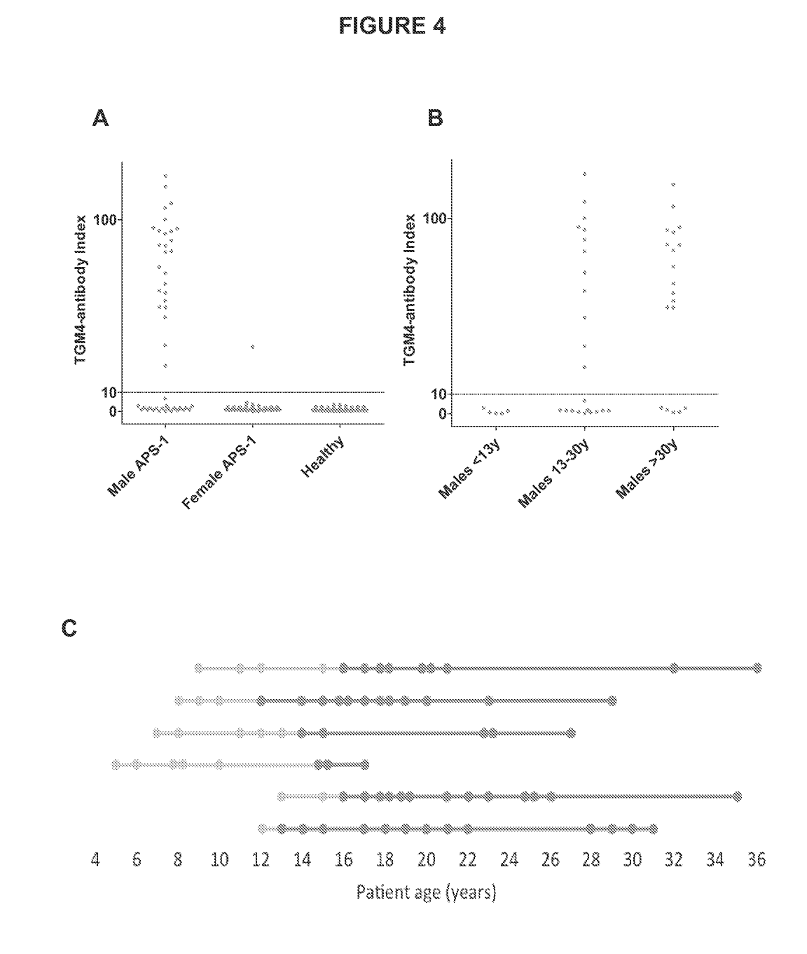

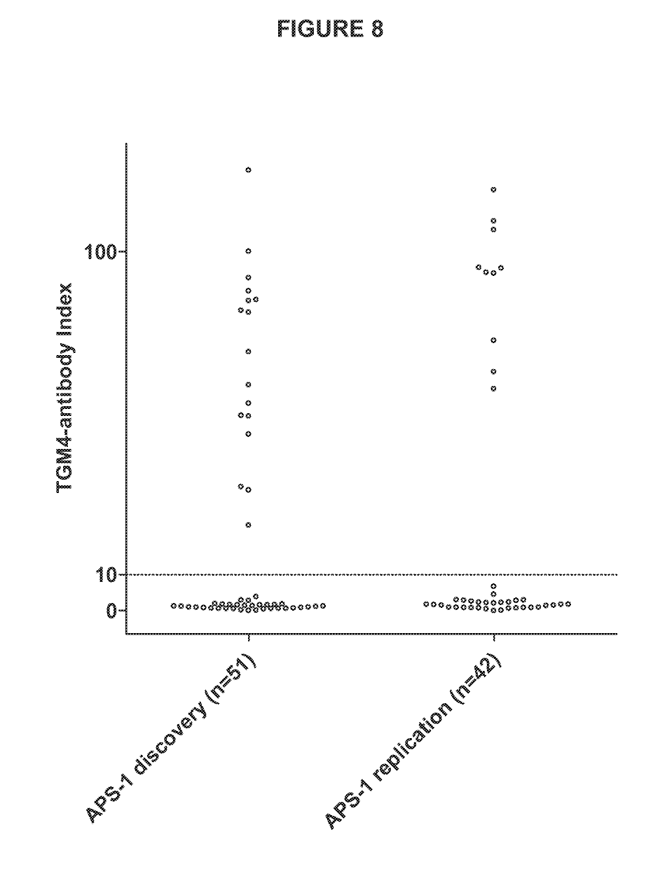

TGM4 autoantibodies are male-specific and appear after the age of pubertal debut. TGM4 is only expressed in the prostate⁷⁻¹⁰ and the inventors therefore compared the frequency of TGM4-specific antibodies between sexes. Among 47 male and 46 female APS-1 patients, the inventors identified 26 reactive males and only one reactive female (FIG. 4A). Furthermore, the TGM4-antibody signal detected in the single positive female was low compared with the males. In the protein array screen, which demonstrated greater dynamic range than the immunopreciptiation assay, the female patient TGM4-antibody signal was only 6% of the TGM4-antibody positive male average (FIG. 8). Consequently, TGM4 autoantibodies were detected in 56% of the males and in one female patient, while strong TGM4-reactivity was exclusively seen among males.

Studies of TGM4 in mouse and other prostate markers in human suggest that TGM4-expression begins during early puberty. Prostate specific antigen (PSA) is not detected in prostate tissue or in the blood of pre-pubertal children, and becomes detectable in tissue by the age of 10 years and a few years later also in blood¹¹⁻¹³. TGM4-expression in the murine prostate commences in parallel with other secretory proteins just before puberty¹⁴. Therefore, the TGM4 autoantibody frequency in different age categories of the male patients was investigated. The prevalence of TGM4 autoantibodies among males over 30 years old (n=19) was 74% and among males at 13-30 years old (n=23) was 52%. However, the five patients younger than 13 years old were all TGM4 autoantibody negative (FIG. 4B). As a comparison the age distribution of established APS-1 autoantigens, including 21-hydroxylase, GAD65, 17-hydroxylase, side chain cleavage enzyme, tyrosine hydroxylase, tryptophan hydroxylase, aromatic L-amino acid decarboxylase and CYP1A2, were analyzed. However all of the established APS-1 autoantigens were represented also in the youngest age category. (Table 2). To further investigate the relation of TGM4 autoantibodies to puberty, consecutive samples from TGM4-reactive patients were analyzed. Six males were selected who were represented by dense consecutive samples from young age. All of the patients could be tracked back to TGM4-seronegativity and they were all confirmed negative up to the age of ten years or younger. TGM4 autoantibodies were first detected by the age of 12, 13, 14, 15, 16 and 16 years respectively and TGM4-reactivity was sustained in the most recent samples taken at the age of 17 to 36 years (FIG. 4C). Importantly, no patients were found to have TGM4 autoantibodies before the age of an expected pubertal debut.

TABLE 2
Male APS-1 patients younger than 13 years old harbor multiple autoantibodies.

| | TGM4 | | | | | |
|---|---|---|---|---|---|---|
| | autoantibody | | Age | Additional | APS-1 | AIRE gene |
| Patients | IP | Array | (yr) | autoantibodies | components | mutations |
| NTF | Neg | Neg | 11 | — | C, HP, HG, | c967-979del13 / |
| | | | | | Al | c967-979del13 |
| F21 | Neg | Neg | 11 | AADC, TH, CYP1A2, | C, M, AH | R257X/R257X |
| | | | | TH | | |
| F24 | Neg | Neg | 11 | 21OH, 17OH | C, HP, AF, | R257X/R257X |
| F35 | Neg | — | 12 | 21OH, 17OH, SCC | C, HP, AF, | R257X/R257X |
| | | | | | IDDM | |
| F36 | Neg | Neg | 10 | GAD65, SCC, DDC, | C, HP, AF, Al | R257X/R257X |
| | | | | TH, TPH | | |

All five male patients younger than 13 years old were negative for autoantibodies to TGM4, as detected by immunoprecipitation (IP) and by the protein array (array). Autoantibodies specific for 21-hydroxylase (21OH), GAD65, 17-hydroxylase (17OH), side chain cleavage enzyme (SCC), tyrosine hydroxylase (TH), tryptophan hydroxylase, aromatic L-amino acid decarboxylase (AADC) and cytochrome P450 1A2 (CYP1A2) were however all represented among the young males. The upper limit of the normal range was defined as an index-value of 10 for the TGM4 immunoprecipitation assay (IP) and as three standard deviations above the average of the healthy controls for the TGM4 protein array results and the other investigated autoantigens. Disease components, including candidiasis (C), hypoparathyroidism (H), adrenal failure (AF), alopecia, hypogonadism (HG) (Al), vitiligo (V), insulin dependent diabetes mellitus (IDDM), malabsortption (M) and pernicious anemia (PA), are presented. Missing data is represented by (—).

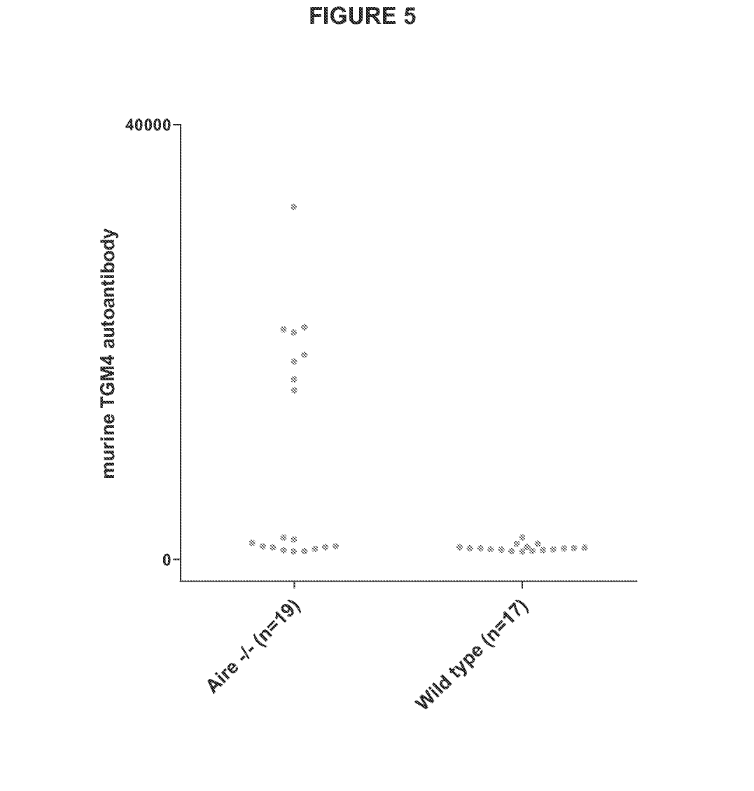

Finally, as shown in FIG. 5, Aire-deficient mice develop lymphocytic infiltration and destruction of the prostate in association with autoantibodies to murine TGM4.

### Example 3

### Discussion

The present inventors report on prostate-specific TGM4 as a major autoantigen in male APS-1 patients, representing the first demonstration of prostate autoimmunity in APS-1. TGM4 autoantibodies were specifically detected in the male and only after the age of pubertal debut. Failure of Aire-dependent immune-tolerance, which may be presumed equal in both sexes, was apparently not sufficient for TGM4-specific autoantibodies to appear. The presentation of antigen in the periphery thus appears to be required for the induction of an autoimmune response.

APS-1 has been appreciated as a model for tissue-specific autoimmune disease and as a system for biomarker discovery. In this study, the inventors screened human protein arrays with APS-1 patient sera to identify new autoantigens and unrecognized target organs. All established autoantigens represented in the array panel were replicated in the screen, and TGM4 was identified as a novel immune target. TGM4 autoantibodies were confirmed by independent methods also in a replication cohort, and were demonstrated APS-1-specific in a large clinical material of patients with different acquired autoimmune diseases. Just more than half of male patients displayed TGM4 autoantibodies, which is in the same range of frequency as the most prevalent tissue-specific autoantigens in APS-1².

TGM4 is a member of the transglutaminase family and is a tissue-specific marker of the prostate. Transglutaminases catalyze a variety of post-translational modifications and have been well recognized for protein cross-linkin^{15-16.} While tissue transglutaminase (TGM2) is ubiquitously expressed, other members exert key functions in specialized tissues and display restricted expression patterns¹⁶. TGM4 is an example of the latter, and is exclusively expressed in the secretory epithelium of the prostate⁷⁻¹⁰. TGM4 is secreted to the prostate lumen and promotes semen coagulation by cross-linking gel forming proteins¹⁷⁻¹⁸.

Members of the transglutaminase family have been identified as target antigens in a diverse group of autoimmune disorders. Celiac disease, as characterized by antibodies specific for tissue transglutaminase 5, may be complicated by manifestations in the skin and nervous system. Dermatitis herpetiformis is associated with antibodies specific for epidermal transglutaminase (TGM3)⁶ while gluten-sensitive cerebellar ataxia and polyneuropathy are linked with antibodies to neuronal TGM6⁴. Acquired FXIII deficient hemophilia is caused by autoantibodies to coagulation factor XIII, which is a hetero-tetrameric protein composed of the two transglutaminase species-F13A1 and F13B3. The recognition of TGM4-specific antibodies in APS-1 further extends the heterogeneity within the family of transglutaminase-autoantibody related diseases.

The current identification of prostate-specific autoantibodies in male patient sera represents the first demonstration of prostate autoimmunity APS-1. Autoantibodies to other tissue specific proteins in APS-1, such as 21-hydroxylase and NALP⁵, are associated with autoimmune destruction of the tissues expressing the antigen^{2,19}. The inventors were, however, unable to investigate the prostate tissue of APS-1 patients, as this could not be ethically motivated. It is interesting to note though, that the Aire-deficient mouse model spontaneously develops lymphocytic infiltration and destruction of the prostate in association with circulating autoantibodies to the prostate epithelium²⁰⁻²². The establishment of a human model for prostate autoimmunity, along with the prostatitis prone Aire-/-mouse, may prove valuable to the understanding of idiopathic prostate disorders such as chronic non-bacterial and inflammatory prostatitis.

TGM4 is the first sex-specific autoantigen described in APS-1 or in any human disease to the inventors' knowledge, and therefore allows a unique observation to be made. As exemplified twice, first by comparing males and females; and second, by comparing males before and after pubertal debut; it was shown that autoantibodies only developed in the presence of the autoantigen. Given that T- and B-cell receptors are generated stochastically, TGM4-reactive lymphocytes are expected to develop in both sexes. TGM4-reactive T-cells may further be expected to escape negative selection equally in the Aire-deficient thymi of male and female patients. However, only in the male and first during puberty would the target antigen be made accessible to the TGM4-reactive lymphocytes, and only then would adequate stimulation provided to trigger an autoantibody response in individuals who had been rendered susceptible.

The mechanisms underlying the development of an autoimmune response are to a great extent unknown, and theories do not always address the autoantigen as an active component. For instance, theories of molecular mimicry imply that autoimmune responses are sufficiently stimulated by an infectious agent and that the autoantigen is merely a passive target of cross-reactivity. These observations of male specific autoimmunity in APS-1 indicate that availability of autoantigen is needed in the periphery for the induction of an autoimmune response.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference:
1 Husebye, E. S., Perheentupa, J., Rautemaa, R. & Kampe, O. Clinical manifestations and management of patients with autoimmune polyendocrine syndrome type I. J Intern Med 265, 514-529 (2009).
2 Soderbergh, A. et al. Prevalence and clinical associations of 10 defined autoantibodies in autoimmune polyendocrine syndrome type I. J Clin Endocrinol Metab 89, 557-562 (2004).
3 Franchini, M., Frattini, F., Crestani, S. & Bonfanti, C. Acquired FXIII inhibitors: a systematic review. J Thromb Thrombolysis 36, 109-114, doi:10.1007/s11239-012-0818-3 (2013).
4 Hadjivassiliou, M. et al. Autoantibodies in gluten ataxia recognize a novel neuronal transglutaminase. Ann Neurol 64, 332-343 (2008).
5 Dieterich, W. et al. Identification of tissue transglutaminase as the autoantigen of celiac disease. Nat Med 3, 797-801 (1997).
6 Sardy, M., Karpati, S., Merkl, B., Paulsson, M. & Smyth, N. Epidermal transglutaminase (TGase 3) is the autoantigen of dermatitis herpetiformis. J Exp Med 195, 747-757 (2002).
7 Dubbink, H. J. et al. Tissue specific and androgen-regulated expression of human prostate-specific transglutaminase. Biochem J 315 (Pt 3), 901-908 (1996).
8 Dubbink, H. J. et al. The human prostate-specific transglutaminase gene (TGM4): genomic organization, tissue-specific expression, and promoter characterization. Genomics 51, 434-444 (1998).
9 Rivera-Gonzalez, G. C. et al. Retinoic acid and androgen receptors combine to achieve tissue specific control of human prostatic transglutaminase expression: a novel regulatory network with broader significance. Nucleic Acids Res 40, 4825-4840 (2012).
10 Su, A. I. et al. A gene atlas of the mouse and human protein-encoding transcriptomes. Proc Natl Acad Sci U S A 101, 6062-6067 (2004).
11 Vieira, J. G., Nishida, S. K., Pereira, A. B., Arraes, R. F. & Verreschi, I. T. Serum levels of prostate-specific antigen in normal boys throughout puberty. J Clin Endocrinol Metab 78, 1185-1187 (1994).
12 Kim, M. R. et al. Serum prostate specific antigen, sex hormone binding globulin and free androgen index as markers of pubertal development in boys. Clin Endocrinol (Oxf) 50, 203-210 (1999).
13 Goldfarb, D. A., Stein, B. S., Shamszadeh, M. & Petersen, R. O. Age-related changes in tissue levels of prostatic acid phosphatase and prostate specific antigen. J Urol 136, 1266-1269 (1986).
14 Pritchard, C. et al. Conserved gene expression programs integrate mammalian prostate development and tumorigenesis. Cancer Res 69, 1739-1747 (2009).
15 Lorand, L. & Graham, R. M. Transglutaminases: crosslinking enzymes with pleiotropic functions. Nat Rev Mol Cell Biol 4, 140-156 (2003).
16 Iismaa, S. E., Mearns, B. M., Lorand, L. & Graham, R. M. Transglutaminases and disease: lessons from genetically engineered mouse models and inherited disorders. Physiol Rev 89, 991-1023 (2009).
17 Malm, J., Hellman, J., Magnusson, H., Laurell', C. B. & Lilja, H. Isolation and characterization of the major gel proteins in human semen, semenogelin I and semenogelin II. Eur J Biochem 238, 48-53 (1996).
18 Dean, M. D. Genetic disruption of the copulatory plug in mice leads to severely reduced fertility. PLoS Genet 9, e1003185 (2013).
19 Alimohammadi, M. et al. Autoimmune polyendocrine syndrome type 1 and NALP5, a parathyroid autoantigen. N Engl J Med 358, 1018-1028 (2008).
20 Hou, Y. et al. An aberrant prostate antigen-specific immune response causes prostatitis in mice and is associated with chronic prostatitis in humans. J Clin Invest 119, 2031-2041 (2009).
21 Ruan, Q. G. et al. The autoimmune regulator directly controls the expression of genes critical for thymic epithelial function. J Immunol 178, 7173-7180 (2007).
22 Setiady, Y. Y. et al. Physiologic self antigens rapidly capacitate autoimmune disease-specific polyclonal CD4+CD25+regulatory T cells. Blood 107, 1056-1062 (2006).
23 Falorni, A., Ortqvist, E., Persson, B. & Lernmark, A. Radioimmunoassays for glutamic acid decarboxylase (GAD65) and GAD65 autoantibodies using ³⁵S or ³H recombinant human ligands. J Immunol Methods 186, 89-99 (1995).
24 Esposito, C. & Caputo, I. Mammalian transglutaminases. Identification of substrates as a key to physiological function and physiopathological relevance. FEBS J 272, 615-631(2005).
25 Grenard, P., Bates, M. K. & Aeschlimann, D. Evolution of transglutaminase genes: identification of a transglutaminase gene cluster on human chromosome 15q15. Structure of the gene encoding transglutaminase X and a novel gene family member, transglutaminase Z. J Biol Chem 276, 33066-33078 (2001).

What is claimed:

1. A method for the diagnosis of autoimmune polyendocrine syndrome type 1, autoimmune prostatitis in autoimmune polyendocrine syndrome type 1, and isolated autoimmune prostatitis comprising detecting autoantibodies specific for TGM4 in a sample obtained from a subject.

2. A method for the treatment of prostate cancer comprising administering a pharmaceutical composition comprising a therapeutically active amount of TGM4 or a fragment thereof to a patient in need of such treatment.

3. A pharmaceutical composition comprising therapeutically effective amount of TGM4 or a fragment thereof for use in the treatment of prostate cancer.

4. Use of TGM4 or a fragment thereof for use in the manufacture of a pharmaceutical composition for treatment of prostate cancer.

5. A method for the treatment of prostate cancer comprising administering a pharmaceutical composition comprising therapeutically effective amount of an antibody, an antibody fragment, a bispecific antibody, or an antibody conjugate specifically or selectively binding to TGM4 to a patient in need of such treatment.

6. A pharmaceutical composition comprising therapeutically effective amount of an antibody, an antibody fragment, a bispecific antibody, or an antibody conjugate specifically or selectively binding to TGM4 for use in the treatment of prostate cancer.

7. Use of an antibody, an antibody fragment, a bispecific antibody, or an antibody conjugate specifically or selectively binding to TGM4 for use in the manufacture of a pharmaceutical composition for treatment of prostate cancer.
